(19) United States
(12) Patent Application Publication
(10) Pub. No.: US 2021/0345878 A1
(43) Pub. Date: Nov. 11, 2021

(54) SYSTEM AND APPARATUS FOR REAL-TIME PUBLIC COMMUNICATION OF HEALTH INFORMATION

(71) Applicant: VitalBadge Corporation, Bellevue, WA (US)

(72) Inventors: Latif Khalil, Bellevue, WA (US); Dennis Batchelder, Bellevue, WA (US)

(73) Assignee: VitalBadge Corporation, Bellevue, WA (US)

(21) Appl. No.: 16/866,867

(22) Filed: May 5, 2020

(51) Int. Cl. A61B 5/00

(52) U.S. Cl. CPC A61B 5/0022; A61B 5/6802; A61B 5/0008; A61B 5/0006

(57) ABSTRACT

Embodiments include apparatuses, methods, and systems of a public communication system including a sensor device, a badge device, a collector device, a server, a smartphone, for monitoring and communicating personal health information. A badge device includes a receiver, a storage unit, and a controller. The receiver is configured to receive, from a sensor device, measured health data of a wearer including a skin temperature of the wearer. The sensor device includes a temperature sensor to measure the skin temperature of the wearer. The storage unit is configured to store health information of the wearer related to the measured health data. The controller is configured to have the health information of the wearer displayed on a display device visible to public, or to initiate a communication to wirelessly transmit the health information of the wearer to another device. Other embodiments may also be described and claimed.

### FIELD

Embodiments of the present disclosure generally relate to the field of public communication of health information.

### BACKGROUND

The background description provided herein is for the purpose of generally presenting the context of the disclosure. Unless otherwise indicated herein, the materials described in this section are not prior art to the claims in this application and are not admitted to be prior art by inclusion in this section.

Wearable Health Devices (WHDs) can continuously monitor human vital signs during daily life, e.g., during work, at home, during sport activities, etc., or in a clinical environment. Hence, WHDs can help people to monitor their health status both at an activity or fitness level for self-health tracking and at a medical level to provide more data to medical personals for diagnostic and guidance of treatment. The advances of semiconductor design and fabrication technology further provide smaller and more reliable WHDs. However, WHDs are primarily designed for personal use and private consumption, and they do not do not address social problems currently experienced.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIGS. 1(a)-1(b) schematically illustrates examples of public communication systems including various devices for monitoring and communicating personal health information, according to various embodiments.

FIG. 2-4 schematically illustrate details of example devices used in a public communication system for monitoring and communicating personal health information, according to various embodiments.

FIG. 5 schematically illustrates a process for monitoring and communicating personal health information in a public communication system, according to various embodiments.

FIG. 6 illustrates a storage medium having instructions for practicing methods described with references to FIGS. 1-5, in accordance with various embodiments.

### DETAILED DESCRIPTION

The following detailed description refers to the accompanying drawings. In the following description, for purposes of explanation and not limitation, specific details are set forth such as particular structures, architectures, interfaces, techniques, etc. in order to provide a thorough understanding of the various aspects of various embodiments. However, it will be apparent to those skilled in the art having the benefit of the present disclosure that the various aspects of the various embodiments may be practiced in other examples that depart from these specific details. In certain instances, descriptions of well-known devices, circuits, and methods are omitted so as not to obscure the description of the various embodiments with unnecessary detail.

People use Wearable Health Devices (WHDs) to collect, analyze, and monitor their health information in order to inform their decisions regarding their health. Medical professionals use WHDs to monitor the health status of their patients in order to inform their diagnostic and treatment decisions about those patients. In both cases, that information is private. However, WHDs are primarily designed for personal use and private consumption. Such WHDs don't address well a need to publicly display human vital signs so they can be interactively used to help build immediate trust with others.

Controlling an outbreak of infectious disease requires identifying and quarantining individuals who are or may be sources of contagion as quickly as possible in order to reduce the spread of the disease. Public health professionals utilize a wide range of established and emerging tools to identify sick people and trace the contacts those people had before learning they were sick in order to prevent people who are or possibly are sources of contagion from contacting other people in order to slow or stop the spread of disease through a population. However, these tools do not generally empower individuals to avoid contact with sick people without avoiding contact much more generally.

Maintaining the social and economic function of a society requires that people have contact with one another. When people may avoid contact with one another out of fear of contracting a disease, this avoidance of contact can make much commercial activity as well as social activity such as attending school, enjoying public places, or attending social events impossible or unacceptably risky. Under such circumstances, a solution that provides people with information about the contemporaneous health status of the individuals with whom they are in proximity is necessary to allow healthy people to avoid contact with sick people in real time. This ability can in turn provide people with the confidence to have contact with others even when communicable disease is present in a population. Embodiments herein includes a system including a public display that helps build immediate trust. In addition, embodiments herein can perform an automatic collection of anonymous health data as well as a list of contacts allows post-fact notifications to the badges who were in previous contact.

Such a solution should provide verifiable, real-time information about the health status of a wearer that can be trusted by those with whom the individual has contact. It should display information about the health of the wearer in a manner that is plainly visible to those around the individual. This health information can be used by a third party to make decisions about his or her own conduct—whether to have contact with the wearer or avoid contact with the wearer, whether to permit the wearer entry into a crowded space or whether to exclude the wearer.

Embodiments herein present a public communication system for personal health information. Herein communication means display and collection, both disassociated from a wearer identity. The public communication system may include a sensor device and a badge device, both wearable by a wearer. The sensor device may be separated from the badge device so that the sensor device can be placed in a body location to obtain more accurate measured health data, e.g., skin temperature, an oxygen saturation level of the wearer, an electrocardiogram (ECG) of the wearer, a respiration rate (RR) of the wearer, or a blood pressure of the wearer. For example, the sensor device may be placed inside a wearer's clothing and in contact with the wearer's armpit, a part of the wearer's upper body, or a part of the wearer's lower body. The sensor device may alternatively be placed in any other appropriate location on the wearer's body. On the other hand, the badge device may be placed around a body location that is easier for public communication. The badge device is defined broadly to be any device that may be attached to any place around a human by any attachment means in location and manner that are readily visible to others in proximity to the user, and the device may be in any shape or form to communicate information, and may not be limited to the shape or form that may be commonly understood as a badge. For example, the badge device may be a badge identifier, a decorative lapel pin, or a necklace, which may be readily visible to members of the public without requiring then to perform any action. A person wearing the sensor device or sensor and badge device may be referred to as a wearer in this document. The badge device may generate health information or determine or display the health status of the wearer based on inputs including measured health data. The health information and the health status may be different from the measured health data. In general, the health information may be generated based on the measured health data to indicate certain health parameters of the wearer, while the health status may be a conclusive information regarding the state of the wearer, e.g., a sick state or a healthy state. For example, when the measured health data is a skin temperature of a wearer measured by a temperature sensor, the health information may be a body temperature generated based on the measured skin temperature. Furthermore, a health status may be an indication whether the wearer is sick or healthy, based on the body temperature, the skin temperature, other health information, and a medical decision policy.

In addition, in embodiments, the health information and/or the health status of a wearer is to be communicated to other wearers or devices, either by visual display or by wireless communication. For example, the health information and/or the health status of the wearer can be displayed on a display device visible to members of the public in order to inform individuals in proximity to the wearer regarding the wearer's health or potential impact on their own health. The display device may also display a social distancing preference set by the wearer. Alternatively, the health information and/or the health status may be broadcasted wirelessly to other devices. Other individuals or organizations may take corresponding actions based on the received health information or health status of the wearer. The health information and/or the health status of the wearer is to be communicated in real time, e.g., within a small predetermined time window. For example, the health information or the health status communicated to the public is determined based on health data measured within a predetermined time period prior to the communication, e.g., 5 minutes to 30 minutes prior to the communication, or within 12 hours of the communication. Hence, the health information or the health status communicated to the public is different from the health update communicated based on a contact tracing or tracking system, where the health update may be communicated days later after an individual has been diagnosed with a communicable disease.

Embodiments herein are further different from currently available WHDs or smart communication devices in many other ways. For the currently available WHDs, the collected data about a wearer is used to inform decisions regarding that wearer. In embodiments disclosed here, the sensor device and the badge device worn by a wearer are used to inform the decisions and impact the behavior of unrelated individuals so that these unrelated individuals can take actions, often not medical actions, to reduce the risks to themselves and others, and further to reassure others to build up trust. Hence, embodiments herein are designed to solve problems, e.g., reduce risk for other wearers, which are different from the problems for currently available WHDs or smart communication devices, e.g., diagnostic or treatment decisions for the same wearer. Accordingly, the health information or the health status of the wearer communicated to other wearers may not need to be as accurate as those health information needed for making diagnostic or treatment decisions for the same wearer.

Embodiments herein present a badge device including a receiver, a storage unit, and a controller coupled with the receiver and the storage unit. The receiver is configured to receive, from a sensor device, measured health data of a wearer including a skin temperature of the wearer. The sensor device includes a temperature sensor to measure the skin temperature of the wearer. The storage unit is configured to store health information of the wearer related to the measured health data. The controller is configured to have the health information of the wearer displayed on a display device visible to public, or to initiate a communication to wirelessly transmit the health information of the wearer to another device.

Embodiments herein present a device including a receiver and a controller coupled to the receiver. The receiver is configured to receive, from a wearable device on a wearer, health information of the wearer related to measured health data including a skin temperature of the wearer. The skin temperature of the wearer is measured by a temperature sensor in contact with the wearer's skin, and is measured within a predetermined time window. The controller is configured to determine a status of the wearer based on the health information of the wearer.

Embodiments herein present a method for monitoring and communicating personal health information in a public communication system. The method includes receiving, from a sensor device, measured health data of a wearer, for example a skin temperature of the wearer, where the sensor device includes a temperature sensor to measure the skin temperature of the wearer. In some embodiments, the measured health data of the wearer may also include a pulse rate of the wearer, an oxygen saturation level of the wearer, an electrocardiogram (ECG) of the wearer, a respiration rate (RR) of the wearer, or a blood pressure of the wearer. The method further includes generating health information of the wearer related to the measured health data, and storing the health information of the wearer. Furthermore, the method includes conveying a health status of the wearer in a manner that is visible to other wearers located in proximity to the wearer, such as within a predefined proximity range, where the health status is related to the health information of the wearer or the measured health data of the wearer. Additionally or alternatively, the method includes transmitting the health information of the wearer to another device.

In the following description, various aspects of the illustrative implementations will be described using terms commonly employed by those skilled in the art to convey the substance of their work to others skilled in the art. However, it will be apparent to those skilled in the art that embodiments of the present disclosure may be practiced with only some of the described aspects. For purposes of explanation, specific numbers, materials, and configurations are set forth in order to provide a thorough understanding of the illustrative implementations. It will be apparent to one skilled in the art that embodiments of the present disclosure may be practiced without the specific details. In other instances, well-known features are omitted or simplified in order not to obscure the illustrative implementations.

Operations of various methods may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiments. Various additional operations may be performed and/or described operations may be omitted, split or combined in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

The description may use perspective-based descriptions such as top/bottom, in/out, over/under, and the like. Such descriptions are merely used to facilitate the discussion and are not intended to restrict the application of embodiments described herein to any particular orientation.

Where the disclosure recites “a” or “a first” element or the equivalent thereof, such disclosure includes one or more such elements, neither requiring nor excluding two or more such elements. Further, ordinal indicators (e.g., first, second or third) for identified elements are used to distinguish between the elements, and do not indicate or imply a required or limited number of such elements, nor do they indicate a particular position or order of such elements unless otherwise specifically stated.

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “coupled with,” along with its derivatives, may be used herein. “Coupled” may mean one or more of the following. “Coupled” may mean that two or more elements are in direct physical or electrical contact. However, “coupled” may also mean that two or more elements indirectly contact each other, but yet still cooperate or interact with each other, and may mean that one or more other elements are coupled or connected between the elements that are said to be coupled with each other. The term “directly coupled” may mean that two or more elements are in direct contact.

As used herein, the term “module” or “unit” may refer to, be part of, or include an Application Specific Integrated Circuit (ASIC), an electronic circuit, a processor (shared, dedicated, or group), and/or memory (shared, dedicated, or group) that execute one or more software or firmware programs, a combinational logic circuit, and/or other suitable components that provide the described functionality.

Various embodiments may include any suitable combination of the above-described embodiments including alternative (or) embodiments of embodiments that are described in conjunctive form (and) above (e.g., the “and” may be “and/or”). Furthermore, some embodiments may include one or more articles of manufacture (e.g., non-transitory computer-readable media) having instructions, stored thereon, that when executed result in actions of any of the above-described embodiments. Moreover, some embodiments may include apparatuses or systems having any suitable means for carrying out the various operations of the above-described embodiments.

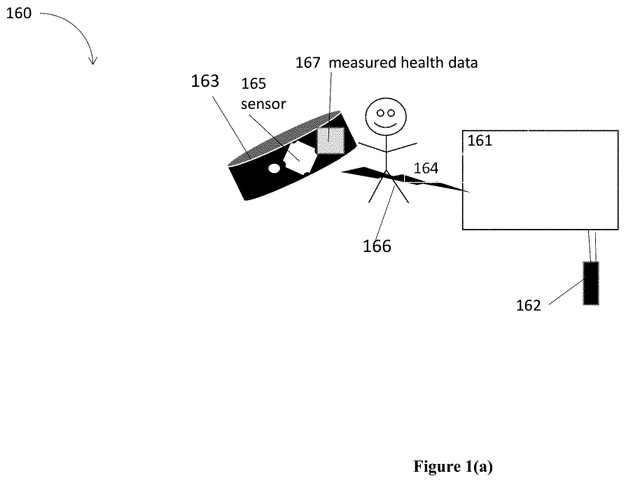

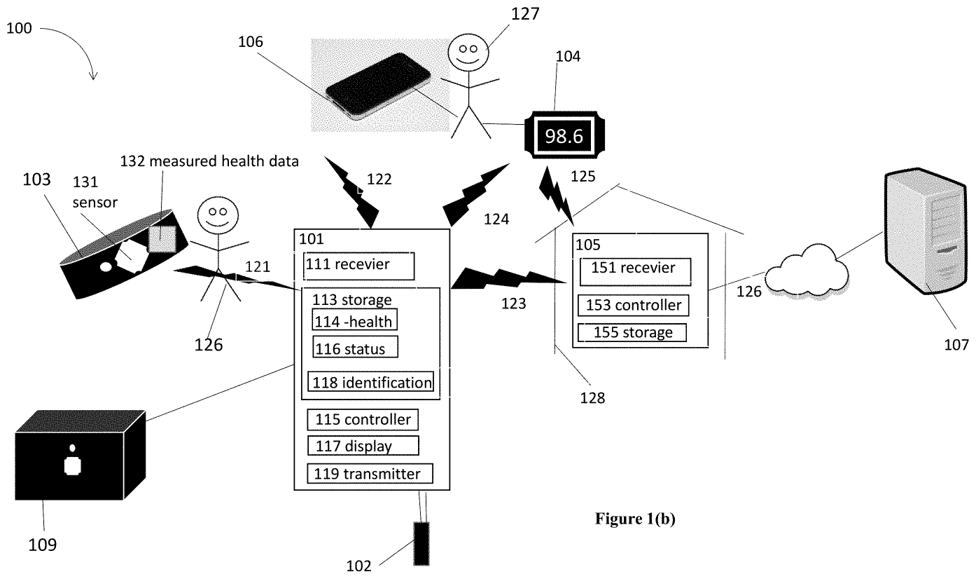

FIGS. 1(a)-1(b) schematically illustrates examples of public communication systems, e.g., a public communication system 100 or a public communication system 160, including various devices for monitoring and communicating personal health information, according to various embodiments.

In embodiments, as shown in FIG. 1(a), the public communication system 160 includes a sensor device 163 and a badge device 161, both worn by a wearer 166. The badge device 161 may include an attachment means 162 to be worn by the wearer 166. There may be a wireless link 164 between the sensor device 163 and the badge device 161, where the wireless link 164 may be a body area wireless network. Furthermore, the sensor device 163 includes a sensor 165 to obtain measured health data 167, which is to be communicated to the badge device 161 through the wireless link 164. The public communication system 160 is just one example. There may be more optional components shown in FIG. 1(b).

In embodiments, as shown in FIG. 1(b), the public communication system 100 includes a sensor device 103, a badge device 101, both worn by a wearer 126. For example, the badge device 101 may include an attachment means 102 to be worn by the wearer 126. The badge device 101 and the sensor device 103 may each be worn in various ways, e.g., attached to a clothes or some special attachments, or directly attached to the body. In addition, the sensor device 103 and the badge device 101 may be recharged or supported by a home unit 109, which may be located in a place accessible by the wearer 126 so that the wearer 126 may recharge the sensor device 103 and the badge device 101, or perform any support operations for the sensor device 103 and the badge device 101.

In embodiments, the public communication system 100 further includes a collector device 105 located in a location 128, a server 107, a second badge device 104 worn by a wearer 127, or a smart device 106 carried by the wearer 127. The public communication system 100 may communicate personal health information among various entities, e.g., the badge device 101, the sensor device 103, the second badge device 104, the smart device 106, the collector device 105, or the server 107.

In embodiments, the sensor device 103 and the badge device 101 may have a wireless link 121 in a first wireless network. The badge device 101 may have a wireless link 123 with the collector device 105, have a wireless link 122 with the smart device 106, and have a wireless link 124 with the second badge device 104. The second badge device 104 may have a wireless link 125 with the collector device 105. The wireless link 122, the wireless link 123, the wireless link 124, and the wireless link 125, may be in a second wireless network, where the first wireless network may be different from the second wireless network. In some embodiments, the first wireless network may be a body area wireless network, and the second wireless network may be a wireless personal area network (WPAN). Furthermore, the collector device 105 may be coupled to the server 107 through a link 126, which may include a wireless or a wired link. In some embodiments, the server 107 may be a cloud server.

The various wireless links, e.g., the wireless link 121, the wireless link 122, the wireless link 123, the wireless link 124, and the wireless link 125, may communicate various data, e.g., measured health data 132, health information of the wearer 114, a health status of the wearer 116. The measured health data of the wearer may include a skin temperature of the wearer, a pulse rate of the wearer, an oxygen saturation level of the wearer, an electrocardiogram (ECG) of the wearer, a respiration rate (RR) of the wearer, or a blood pressure of the wearer. More details of the health information of the wearer 114 and the health status of the wearer 116 are discussed in paragraphs below.

In embodiments, the sensor device 103 includes a temperature sensor 131 to measure the skin temperature of the wearer 126, which produces a measured health data 132 for the wearer 126. The measured health data 132 of the wearer 126 may further include a pulse rate of the wearer 126, an oxygen saturation level of the wearer 126, an electrocardiogram (ECG) of the wearer 126, a respiration rate (RR) of the wearer 126, a blood pressure of the wearer 126, or some other measured health data of the wearer 126 obtained by different sensors. The temperature sensor 131 may be located inside the wearer's clothing and in contact with the wearer's armpit, a part of the wearer's upper body, a part of the wearer's lower body, or the wearer's forehead, hands, or any other suitable body part. In some embodiments, the sensor device 103 may be a ring to be worn around a finger of the wearer 126. The temperature sensor 131 may include a resistive temperature detector, or a negative temperature coefficient thermistor. The temperature sensor 131 is used as an example. In some other embodiments, other kinds of sensors may be used, with or without a temperature sensor.

In embodiments, the badge device 101 may be any item that is worn or otherwise affixed to the wearer or the wearer's clothing in a readily visible location, such as a badge identifier, a decorative lapel pin, or a necklace. A wearer may be whoever happens to be wearing the badge device 101. The system 100 does not keep track of who is wearing the badge device 101, and does not tie the badge device 101 to a wearer profile or wearer identity. The badge device 101 includes a receiver 111, a storage unit 113, a controller 115, a display device 117, and a transmitter 119. The receiver 111 is to receive, from the sensor device 103, the measured health data 132 of the wearer. The controller 115 is configured to generate and display health information 114 and/or a health status 116 of the wearer based on the measured health data 132. In general, a health information may be generated based on the measured health data to indicate certain health parameters of the wearer, while a health status may be information regarding the state of the wearer, e.g., a sick state, a healthy state, an immune state, or an unknown state. Health status, e.g., a sick state, a healthy state, an immune state, or an unknown state, may be presumed states, and the badge device 101 may make no decision on the actual status of the wearer.

In some embodiments, the health status may be a pre-defined level of risk associated with the wearer. For example, when the measured health data 132 is a skin temperature of the wearer 126 measured by the temperature sensor 131, the health information 114 may be a body temperature generated based on the measured health data 132, e.g., measured skin temperature. Furthermore, the health status 116 may be an indication whether the wearer 126 may be exhibiting symptoms of illness, based on the body temperature, the skin temperature, or other any other health information appropriate, such as verified immunity from disease conferred by recovery or vaccination. The health status may be associated with pre-defined states or risk levels. In some embodiments, there may be only one type of health information 114 or health status 116.

The body of a person is typically divided into two regions—the core and the shell (including skin and extremities). The body is always trying to maintain an ideal temperature range in the core. On the other hand, the range of the temperature at the shell fluctuates more widely. For example, the core body temperature might be relatively stable while the skin temperature changes based on the environment and the body's internal adjustments to make the person warmer or colder. As a result, skin temperature will vary depending on circumstances and the location one of the measurement. The core body temperature is the clinical standard, which may be used for diagnostic or treatment decisions for the person. Measuring core body temperature generally requires invasive methods of measurement. When core temperature is measured for clinical purposes, shell temperature is measured in a non-invasive site (e.g., mouth, armpit, the tympanic membrane, the temporal artery, or rectum), and that measurement is used to provide an estimation of core temperature. Shell temperature, e.g., the skin temperature, is less invasive to measure and can provide insights on the same rhythms as core temperature—menstrual cycles, daily fluctuations, circadian rhythms, etc.—as well as other health indicators like a fever. In embodiments, the separation of the sensor device 103 and the badge device 101 can help to obtain more accurate skin temperature at desired locations, e.g., the armpit, the forehead, the ear, the hand, etc. The controller may generate health information or determine health status based on a single measurement of temperature or on multiple measurements, and may generate health information or determine health status based on change in temperature over a pre-determined time interval. Additionally, the controller may use measurement of or information about the ambient temperature or other ambient environmental conditions in its generation of health information and/or determination of health status. Additionally, the controller may estimate body temperature within a range, or may determine body temperature within a certain predetermined level of accuracy.

In embodiments, the storage unit 113 is configured to store the health information 114 and/or the health status 116 of the wearer 126 related to the measured health data 132. In addition, the storage unit 113 is further configured to store an identifier 118 of the badge device 101. The identifier 118 may be an identifier assigned to the badge device 101, having no connection to any other personal information of the wearer 126. For example, the identification 118 may be independent from a cellular phone number of a phone identification number of the wearer 126. By storing an independent identifier 118, security and privacy for the wearer 126 may be achieved even in a public communication system 100 since the identifier 118 is not linked to any other personal information of the wearer. The identifier 118 may be text, a number, or any other identification mechanism capable of distinguishing information or transmissions from one badge device from any other badge device.

In embodiments, the controller 115 is further configured to have the health information 114 or the health information 116 of the wearer displayed on the display device 117 visible to public, or to initiate a communication to wirelessly transmit the health information 114 or the health information 116 of the wearer to another device by the transmitter 119. The display device 117 is coupled to the controller 115 and the storage unit 113, and is configured to convey the health status 116 or the health information 114 in a manner that is visible to other persons, e.g., a member of the public 127, located within proximity to the wearer, such as within a predefined proximity range. The health status 116 may be related to the health information 114 of the wearer or the measured health data 132 of the wearer 126. In some embodiments, the health status may be based on previously measured health data. For example, a wearer may have a fever, resulting in a health status of sick until the wearer's body temperature has been normal for 48 hours. Temperature readings along with the time and date taken may be stored over time so that the controller may determine when the individual has been fever-free for 48 hours, at which point the health status may be changed. In some embodiments, the display device 117 is configured to convey the health information 114 or the health information 116 of the wearer 126 by audio or visual cues including readable digits, color based visual alters, color patterns, sounds, or audio alerts.

In some embodiments, the badge device 101 may be a badge identifier that can display on the display device 117 the wearer's health information 114 or the health status 116. The health information 114 or the health status 116 displayed on the display device 117 may be determined in real time, e.g., based on health data measured within a short time of the determination and display, such as within a predefined window of 1 minute to 30 minutes before display, or within the same 12 hours window before display. The health information 114 or the health status 116 may be disassociate with other personal information, e.g., personally identifying information such as name, or phone number, driver license, and any other personal information.

In some embodiments, the health information 114 or the health status 116 may be displayed as text including characters and numerals indicating the body temperature or health status of the wearer 126. For example, the health status 116 may be displayed as a healthy state, or a sick state, of the wearer 126, based on the measured health data 132 or other inputs. When the measured health data 132 falls outside a predetermined range, e.g., a temperature is higher than a predetermined threshold value, e.g., 101 degrees, the display device 117 may display a text, or display a bright color pattern, visual and audio alerts, for others to see and recognize the displayed information to determine the health status, e.g., in a healthy state or a sick state, of the wearer 126. For example, the display device 117 may be in an on state, followed by an off state, followed by an on state, to drop attention from the wearer 127 or other persons in the location 128. The sensor device 103 and the badge device 101 are not tied to person 126 in any way. Rather, the sensor device 103 and the badge device 101 could be used by another person at any time.

In embodiments, the health information 114 or the health status 116 may be transmitted by the transmitter 119 to the second badge device 104 by the wireless link 124. The health information 114 or the health status 116 transmitted by the transmitter 119 may be determined in real time, e.g., based on health data measured within 1 minute to 30 minutes of the transmission, or within the same 12 hours before they are transmitted. The wearer 126 may allow the badge device 101 to share health information 114 or the health status 116 with other badge devices individually, in a group, or to anyone in a configurable proximity.

In embodiments, the receiver 111 is configured to receive continuously or substantially continuously from the sensor device 103 the measured health data 132. For example, the receiver 111 is configured to receive multiple types of measured health data of the wearer at multiple time instances within a short period of time, such as six times per minute, and to contemporaneously update the displayed health information in response to any changes in measured health data such that the displayed health information reflects the current state of the wearer. The storage unit 113 is configured to store multiple types of health information of the wearer related to the multiple types of measured health data. The controller is configured to have the multiple types of health information of the wearer displayed on the display device at multiple time instances, or to initiate the communication to wirelessly transmit the multiple types of health information of the wearer to another device.

In embodiments, when the badge device 101 indicates that the wearer 126 is in a healthy or asymptomatic state, and the second badge device 104 indicates that the wearer 127 is in a healthy state, the wearer 126 wearing the badge device 101, the wearer 127 wearing the second badge device 104, or more wearers who are also in a healthy or asymptomatic state may form a dynamic group or circle of proximity trust within which the uses can interact with each other having an increased feeling of safety. Therefore, the badge device 101 and the second badge device 104 provide an efficient way to build trust that there is a reduced risk of transmission of infectious disease between individual wearers. The wearer 127 may approach the wearer 126, and before she gets too close, the wearer 127 may look at the badge device 101 worn by the wearer 126 and be alerted that the wearer 126 may be experiencing symptoms of illness as indicated by the message displayed in the display device 117. The wearer 127 may keep her distance from the wearer 126 and reduce the risk of any communicable virus or disease, e.g., COVID-19 virus. Accordingly, the badge device 101 worn by the wearer 126 may help manage very large events and activities, e.g., concerts and theme parks, where multiple people may interact with each other. For example, a person may have the ability to reduce their own health risks by the health information 114 or the health status 116 displayed on a display device of the badge device 101 worn by other people, e.g., people in home deliveries, in-home services (e.g., cleaners, workers, health professionals), casual public meetings, group exercises, family and neighborhood gatherings, meetups, or any other group gatherings.

In embodiments, the badge device 101 worn by the wearer 126 may also exchange other information, e.g., the identification 118, with the second badge device 104. For example, the wearer 126 and the wearer 127 get “in-contact” (within close proximity) with each other. While they are in-contact, the badge device 101 and the second badge device 104 may exchange the identifiers of each device, and record the date/time as well as the duration of each in-contact session (“in-contact information”). This information may be used to allow authorities and members of the public to determine with whom infected people were in contact while protecting wearer privacy.

In embodiments, the badge device 101 is verified to be authentic and operational. The badge device 101 may respond to a challenge so others can verify the badge device 101 is authentic and operational. When a badge device is authentic and operational, the information displayed on the display is reasonably reliable and reasonably safe from manipulation or selective transmission or display by the wearer. For example, the wearer 127 may type in the second badge device 104 identifying number, a visual indication may appear in response on the second badge device, affirming that the second badge device is operational.

In addition, the wearer 126 may set his social distance preference, and this would display on the badge device 101, and be collected by collector 105/exchanged with other badge devices, e.g., the second badge device 104, along with the health information, and would be visible to others. For instance, a wearer could set to: (1) please keep your distance, (2) I feel safe for close contact, (3) ask me first, or (4) I have no social distance preference. Such wearer-decided messages displayed on the badge device 101 may give others an unspoken social cue as to what is tolerable. It enables them to protect themselves using a badge device even if the wearer 126 has the only badge device in the room: others know to keep their distance/come close without asking, and it helps vulnerable people let others know they need their cooperation to stay safe.

In embodiments, the collector device 105 is located in the location 128 and includes a receiver 151, a controller 153, and a storage unit 155. The receiver 151 is to receive, from a wearable device on a wearer, e.g., the badge device 101, the health information 114 or the health information 116 of the wearer related to the measured health data 132. The measured health data 132 may include a skin temperature of the wearer 126 measured by the temperature sensor 131 within a predetermined time window. The controller 153 is configured to determine a status of the wearer 126 based on the health information 114 or the health information 116 of the wearer 126. The storage device 155 is coupled to the controller 153 and the receiver 151, where the storage device 155 is configured to store the received health information or health status.

In some embodiments, the determined status of the wearer by the collector device 105 may be the same as the health status 116 stored in the badge device 101. In some other embodiments, the status of the wearer determined by the controller 153 may be different from the health status 116. For example, the wearer 126 may have a healthy state as the health status 116, however, the collector device 105 may determine that the wearer may still have high risk due to some health information, e.g., having some previous contact with some other people who are sick. In some other embodiments, the status determined by the collector device 105 may be a status to admit the wearer 126 or deny the wearer 126 to the location 128, or a status related to actions to take place in the location 128.

In embodiments, the location 128 may be a government or commercial office that may require badged entry. The location 128 may be schools, daycare centers, college campus, airport waiting lounges, business premises, or other places occupied by a group of people. The collector device 105 can save time for manual inspection of the badge device worn by a wearer, and further decrease risks for the people in location 128.

In embodiments, the controller 153 may be further configured to alter people within a predetermined range of a health status of the wearer 126. In addition, the controller 153 is configured to send the received health information 114 or the received health status 116 of the wearer 126 to a network device, e.g., the server 107. The controller 153 may be configured to receive commands from the network device e.g., the server 107. In embodiments, the collector device 105 may further include a video camera. The controller 153 is to activate the video camera to capture information about the wearer 126 having the wearable device based on the received health information 114 of the wearer.

In embodiments, the collector device 105 is located in a business premises to screen visitors. For example, a bar may require all employees and customers to wear the badge device 101 when they are on the premises. When a first customer comes to the bar wearing the badge device 101, the collector device 105 may determine the first customer is in a healthy state based on the health information 114 or the health information 116 of the wearer received by the receiver 151, and determine the first customer is admissible to the bar. A second customer comes to the bar wearing the badge device 101, the collector device 105 may determine the second customer is in a sick state based on the health information 114 or the health information 116 of the wearer received by the receiver 151, and deny the second customer from entering the bar. A third customer is admitted to the bar when he comes in a healthy state. However, after he enters the bar, his temperature may rise until it exceeds a predetermined threshold, and the badge device 101 may determine based on health information 114 or the health information 116 that he become sick while he was in the bar. The collector device 105 may alert bar staff that a customer in the bar is exhibiting signs of illness so that the bar staff can react accordingly, such as by removing the customer from the bar.

In embodiments, the location 128 may be a hospital patient visiting room and the collector device 105 is located in the visiting room. All visitors to the visiting room have to wear the badge device 101. The collector device 105 may determine a status of a visitor based on the health information or the health status of the visitor received by the receiver 151 of the collector device 105. Medical staff may adjust or updated risks of each visitor. For example, the staff may change patient priorities, move people to isolation zones, and schedule disinfection cleaning.

In embodiments, the location 128 may be a nursing home and the collector device 105 is located in the nursing home. All residents of the nursing home may wear the badge device 101. The collector device 105 may determine a status of the residents based on the health information or the health status of the residents received by the receiver 151 of the collector device 105. Hence, the nursing home may manage risks, including isolating residents, scheduling disinfection cleaning, and limiting visitors.

In embodiments, the collector device 105 may be located in a public location to collect information other than health information or the health status of the visitors. For example, the collector device 105 may collect the identification of the badge device 101 worn by each visitor. The identification of the badge device may be provided to the collector device 105 in an encrypted way.

In some embodiments, the collector device 105 may be implemented in software, e.g., an application running on a smartphone. When the collector device 105 is an application, the collector device 105 may not decrypt nor retain the received health information or the health status of the visitors, but to further send the received health information or the health status of the visitors to the server 107 for decryption.

In embodiments, the server 107 may be located remotely in a cloud to processes data provided by the collector device 105. For example, the server 107 may assess different risk levels for different wearers based on the health information or the health status from the wearers. For example, a first wearer may have a higher risk level since the first wearer was in contact with a known sick person. A second wearer may have a lower risk level since the second wearer is immune to a virus, e.g., COVID-19 virus. The server 107 may produce a list of assessments for a group of wearers according to the badge device identification, the date/time and length assessment should be active. The list of assessments may be updated when new information arrives.

In embodiments, the server 107 may respond to a query from the collector device 105 to send a history of health information or health status of a wearer based on the badge device worn by the wearer. Such a history of health information or health status may not be saved in the collector device 105 but provided to the collector device 105 upon request. In some embodiments, the server 107 may determine a wearer is sick based on the recent collected health information from the collector device 105 and inform the collector device 105 that the wearer may be sick.

In embodiments, the communication between the server 107 and the collector device 105 may be conducted without a smartphone. No location information may be collected. The communication between the server 107 and the collector device 105 may be conducted without the collector device 105 knowing the identification information of the badge device for the wearer. Therefore, the communication between the server 107 and the collector device 105 is secure for the wearer.

In embodiments, the home unit 109 may include a rechargeable power source that can be used to recharge the badge device 101 and the sensor device 103 individually or together. In some embodiments, the charging of the badge device 101 and the sensor device 103 may be done by wireless technology. The home unit 109 may also include a standard recharging connector, such as USB-C, Micro-USB, and Lightning, so it can be recharged using the wearer's existing power options they use for their smartphones. In addition, the home unit 109 may be able to be configured, setup, and update firmware in the badge device 101 and the sensor device 103. The home unit 109 may further include, optionally, a clock, a local storage, and an ability to securely download/upload data to an optional smartphone Application.

In embodiments, the smartphone 106 may have a badge device application. The badge device application may include the ability to pair with one or more other badge devices, the ability to display the health information or health status of other proximate wearers, ability to join or leave a managed group for special use cases such as public health and community health tracking purposes; and the ability o proxy data from third party sensor devices to a badge device.

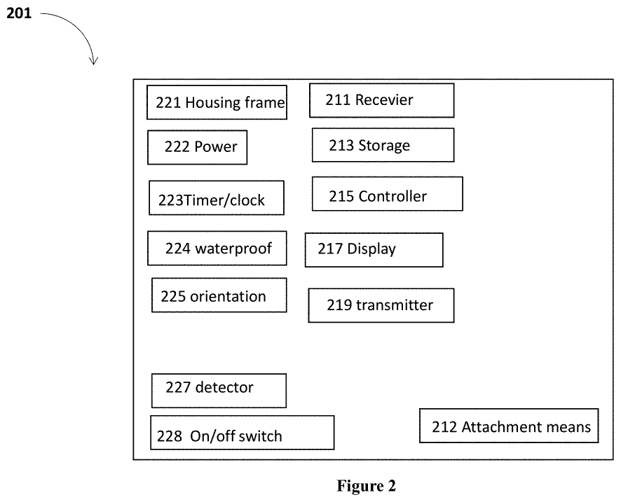

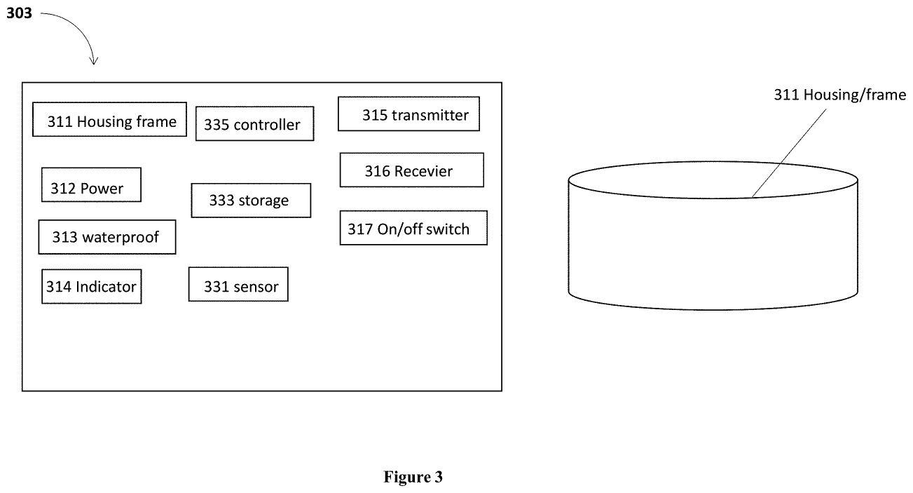

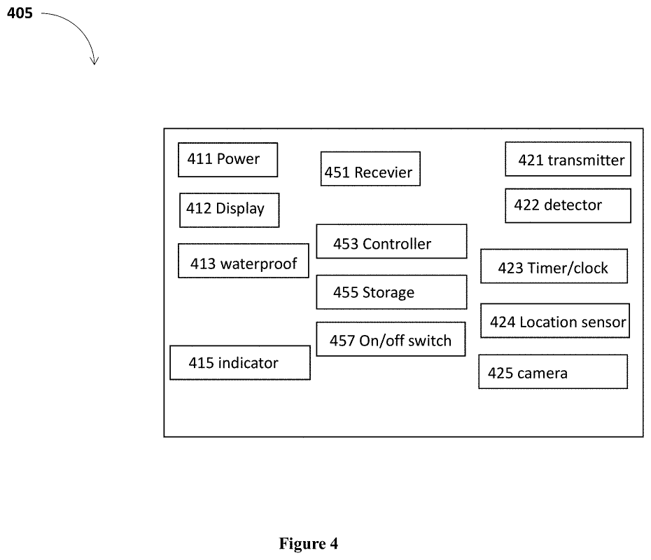

FIG. 2-4 schematically illustrate details of example devices for monitoring and communicating personal health information, according to various embodiments. FIG. 2 illustrates an example badge device 201, FIG. 3 illustrates an example sensor device 303, and FIG. 4 illustrates an example collector device 405, which may be examples of the badge device 101, the sensor device 103, and the collector device 105, as shown in FIG. 1.

In embodiments, as shown in FIG. 2, the badge device 201 includes a receiver 211, an attachment means 212, a storage unit 213, a controller 215, a display device 217, and a transmitter 219, which may be similar to the receiver 111, the attachment means 102, the storage unit 113, the controller 115, the display device 117, and the transmitter 119, respectively. The display device 217 may be capable of showing the health information received from a sensor device or other sources. The display device 217 can be set to show digital information, color code/patterns, or to produce audio and haptic signals. The storage unit 213 may save and keep track of recent history of health information, health data, or health status. The transmitter 119 can provide health information or health status to other badge devices or collector devices in proximity.

In addition, the badge device 201 may include a housing frame 221, a power source 222, a timer/clock 223, a waterproof cover 224, an orientation indicator 225, a detector 227, and an on/off switch 228. The power source 222 is to provide power for the display device 217 and communications. In some embodiments, the power source 222 may be a rechargeable battery that recharges wirelessly through a home unit, e.g., the home unit 109. The badge device 201 includes the waterproof cover 224 so that the badge device 201 can be worn in high moisture environments. The badge device 201 uses the detector 227 to detect other proximate badge devices. The controller 215 is configured to exchange (receive and transmit) health and proximity data with other badge devices, wearable health devices, and smart phones. The on/off switch 228 can turn the badge device 201 on or off, show/change current state, such as digital display, color codes, patterns, temperature units, share data with others, visual/audio signals, notifications/alarms, etc. The orientation indicator 225 can automatically detect that the badge device 201 is worn correctly with correct orientation.

In embodiments, the badge device 201 may securely, reliably, and continuously receive health data (temperature, pulse, etc.) and context data (such as location, speed) from a sensor device, a smartphone, or other third party sensors. The badge device 201 may also initiate a challenge and respond to challenges to/from other badge devices or collector devices nearby. The badge device 201 may further notify the wearer if it loses contact with its sensor device, a smartphone, or another badge device. The notification to the wearer may be a visual, audio, or haptic notification.

In embodiments, the wearer of the badge device may have full control over display and sharing of his/her data with other badge devices. The badge device 201 may be used as a personal health identification, like a badge with Radio-frequency identification (RFID), to allow/deny a wearer to enter a public setting such as offices, events, shows, restaurants, social gathers, schools, churches, entertainment parks, etc. A public place may deploy a collector device to gather health data from the badge device 201.

In embodiments, as shown in FIG. 3, the sensor device 303 includes one or more sensors 331, a storage unit 333, a controller 335, a receiver 316, a transmitter 315, an on/off switch 317, a housing frame 311, a power source 312, a waterproof cover 313, and an indicator 314. The power source 312 is to provide power to the sensor device 303. In embodiments, the power source 312 may be a rechargeable battery that recharges wirelessly through the home unit. The sensor device 303 may have the waterproof cover 313 so that the sensor device 303 can be worn in high moisture environments and even in the water. The sensor or sensors 331 may continuously measure health data such as skin temperature, pulse, oxygen levels, etc. In embodiments, the sensor 331 may be in continuous contact with the wearer's skin while in use. The sensor device 303 may securely and continuously send, by the transmitter 315, the currently measured health data to its badge device. The housing frame 311 may be a durable housing and may be of various shapes, e.g., circle, rectangle, or other shapes. The sensor device 303 may be a ring, wrist/leg/head bands, necklaces, or disposable patches, or other wearable sensors. The sensor 331 may be detached from the housing frame 311 so that the wearer can replace, modify, or design their own appropriate housing. The Indicator 314 may illustrate the connection status with the badge device, power level, and whether the sensor device 303 is properly situated to collect health data.

In embodiments, as shown in FIG. 4, the collector device 405 includes a receiver 451, a controller 453, a storage unit 455, an on/off switch 457, a transmitter 421, a detector 422, a power source 411, a display device 412, a waterproof cover 413, an indicator 415, a timer/clock 423, a location sensor 424, and a video camera 425. The power source 411 may be rechargeable batteries for backup. The waterproof cover 413 may enable the collector device 405 to be placed outside a building. The receiver 451 may receive or collect health data from proximate badge devices. The location sensor 424 may be a GPS sensor. The controller 453 may activate the video camera 425 to capture information about the wearer having the wearable device based on the received health information of the wearer. The video camera 435 may also capture more information about people passing by the collector device 405, when the health information of the wearer may indicate a high risk health status. The indicator 415 is to show the collector device 405 is functioning properly. The storage unit 455 may be used to store gathered data and video. The transmitter 421 may send collected data to a server, and to receive commands from the server. In addition, a visual and/or audio alter may be used to show a public alert when the collector device 405 detects a wearer having a high risk health status based on the health information or health status received from a badge device worn by the wearer.

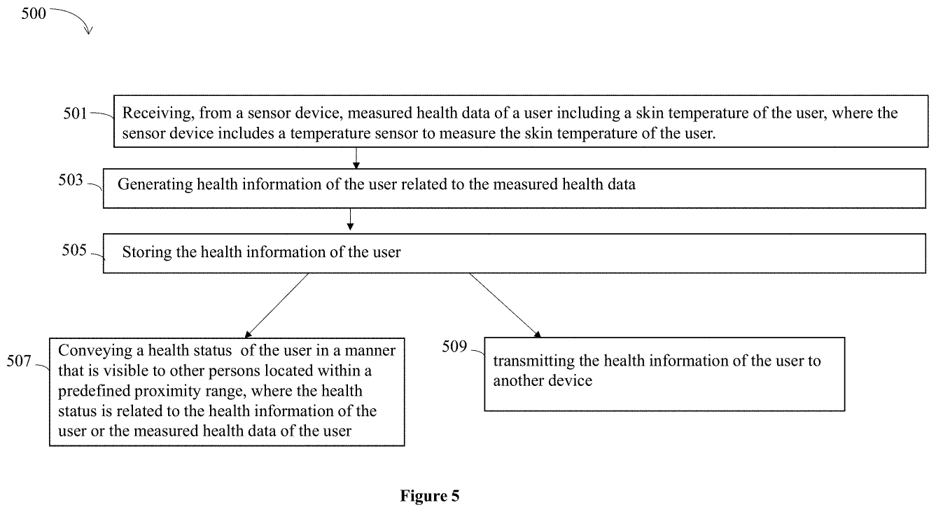

FIG. 5 schematically illustrates a process 500 for monitoring and communicating personal health information in a public communication system, according to various embodiments. In embodiments, the process 500 may be performed by the badge device 101 or the badge device 201 as shown in FIGS. 1-2.

At a block 501, the process 500 may include receiving, from a sensor device, measured health data of a wearer including a skin temperature of the wearer, where the sensor device includes a temperature sensor to measure the skin temperature of the wearer. For example, as shown in FIG. 1, at the block 501, the process 500 may include receiving, from the sensor device 103, measured health data 132 of the wearer 126 including a skin temperature of the wearer, where the sensor device 103 includes a temperature sensor 131 to measure the skin temperature of the wearer 126.

At a block 503, the process 500 may include generating health information of the wearer related to the measured health data. For example, as shown in FIG. 1, at the block 503, the process 500 may include generating health information 114 of the wearer 126 related to the measured health data 132.

At a block 505, the process 500 may include storing the health information of the wearer. For example, as shown in FIG. 1, at the block 505, the process 500 may include storing the health information 114 of the wearer 126 in the storage device 113.

At a block 507, the process 500 may include conveying a health status of the wearer in a manner that is visible to other persons located within a predefined proximity range, where the health status is related to the health information of the wearer or the measured health data of the wearer. For example, as shown in FIG. 1, at the block 507, the process 500 may include conveying the health status 116 of the wearer 126 in a manner that is visible to other persons located within a predefined proximity range, where the health status 116 is related to the health information 114 of the wearer or the measured health data 132 of the wearer.

At a block 509, the process 500 may include transmitting the health information of the wearer to another device. For example, as shown in FIG. 1, at the block 507, the process 500 includes transmitting the health information 114 of the wearer to the second badge device 104.

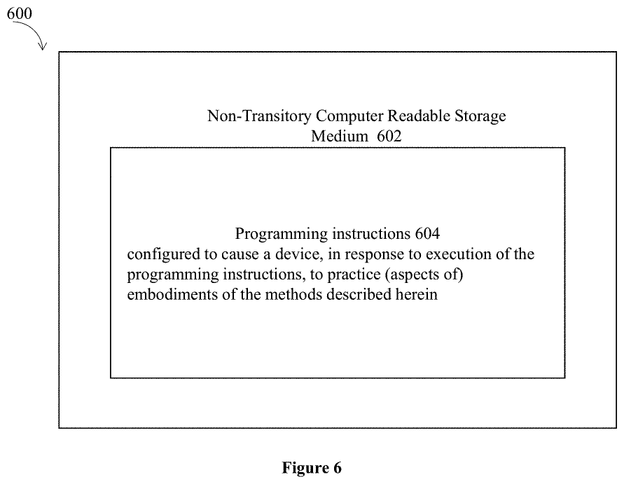

Furthermore, the present disclosure may take the form of a computer program product embodied in any tangible or non-transitory medium of expression having computer-usable program code embodied in the medium. FIG. 6 illustrates an example computer-readable non-transitory storage medium that may be suitable for use to store instructions that cause an apparatus, in response to execution of the instructions by the apparatus, to practice selected aspects of the present disclosure. As shown, non-transitory computer-readable storage medium 602 may include a number of programming instructions 604. Programming instructions 604 may be configured to enable a device, in response to execution of the programming instructions, to perform, e.g., various operations associated with the process 500 for monitoring and communicating personal health information in a public communication system. The various operations may be performed by the sensor device 103, the badge device 101, the collector device 105, the server 107, the second badge device 104, or the smart device 106, as shown in FIG. 1.

In alternate embodiments, programming instructions 604 may be disposed on multiple computer-readable non-transitory storage media 602 instead. In alternate embodiments, programming instructions 604 may be disposed on computer-readable transitory storage media 602, such as, signals. Any combination of one or more computer usable or computer readable medium(s) may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the wearer's computer, partly on the wearer's computer, as a stand-alone software package, partly on the wearer's computer and partly on a remote computer or entirely on the remote computer or server. In the latter scenario, the remote computer may be connected to the wearer's computer through any type of network, including a local area network (LAN) or a wide area network (WAN), or the connection may be made to an external computer (for example, through the Internet using an Internet Service Provider).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions. As used herein, “computer-implemented method” may refer to any method executed by one or more processors, a computer system having one or more processors, a mobile device such as a smartphone (which may include one or more processors), a tablet, a laptop computer, a set-top box, a gaming console, and so forth.

Embodiments may be implemented as a computer process, a computing system or as an article of manufacture such as a computer program product of computer readable media. The computer program product may be a computer storage medium readable by a computer system and encoding a computer program instructions for executing a computer process.

The corresponding structures, material, acts, and equivalents of all means or steps plus function elements in the claims below are intended to include any structure, material or act for performing the function in combination with other claimed elements are specifically claimed. The description of the present disclosure has been presented for purposes of illustration and description, but is not intended to be exhaustive or limited to the disclosure in the form disclosed. Many modifications and variations will be apparent to those of ordinary skill without departing from the scope and spirit of the disclosure. The embodiment are chosen and described in order to best explain the principles of the disclosure and the practical application, and to enable others of ordinary skill in the art to understand the disclosure for embodiments with various modifications as are suited to the particular use contemplated.

Thus various example embodiments of the present disclosure have been described including, but are not limited to:

The foregoing description of one or more implementations provides illustration and description, but is not intended to be exhaustive or to limit the scope of embodiments to the precise form disclosed. Modifications and variations are possible in light of the above teachings or may be acquired from practice of various embodiments.

These modifications may be made to the disclosure in light of the above detailed description. The terms used in the following claims should not be construed to limit the disclosure to the specific implementation disclosed in the specification and the claims. Rather, the scope of the disclosure is to be determined entirely by the following claims, which are to be construed in accordance with established doctrines of claim interpretation.

What is claimed is:

1. A device, comprising:
a receiver to receive, from a sensor device, measured health data of a wearer;
a storage unit configured to store health information of the wearer related to the measured health data; and
a controller coupled to the storage unit and the receiver, and configured to have the health information of the wearer displayed on a display device visible to public, and to initiate a communication to wirelessly transmit the health information of the wearer to another device.

2. The device of claim 1, wherein the health information of the wearer includes a body temperature of the wearer determined based on the skin temperature of the wearer measured by the temperature sensor.

3. The device of claim 1, wherein the controller is configured to generate the health information of the wearer based on the measured health data.

4. The device of claim 1, wherein the measured health data of the wearer further includes a pulse rate of the wearer, an oxygen saturation level of the wearer, an electrocardiogram (ECG) of the wearer, a respiration rate (RR) of the wearer, or a blood pressure of the wearer.

5. The device of claim 1, wherein the temperature sensor is located inside a wearer's clothing and in contact with the wearer's armpit, a part of the wearer's upper body, or a part of the wearer's lower body.

6. The device of claim 1, wherein the temperature sensor includes a resistive temperature detector, or a negative temperature coefficient thermistor.

7. The device of claim 1, further comprising:
a display device coupled to the controller and the storage unit, and the display device is configured to convey a health status in a manner that is visible to other persons located within a predefined proximity range, wherein the health status is related to the health information of the wearer or the measured health data of the wearer.

8. The device of claim 7, wherein the display device is configured to convey the health status of the wearer by audio or visual cues including readable digits, color based visual alters, color patterns, sounds, or audio alerts.

9. The device of claim 1, wherein the display device is configured to indicate a social distance preference.

10. The device of claim 1, wherein the receiver is configured to receive the measured health data from the sensor device in a first wireless network, and the device further includes a transmitter to transmit the health information of the wearer to another device in a second wireless network, wherein the first wireless network is different from the second wireless network.

11. The device of claim 10, wherein the first wireless network is a body area wireless network, the second wireless network is a wireless personal area network (WPAN), and the transmitter is to transmit the health information of the wearer to another device by broadcasting.

12. The device of claim 1, wherein:
the receiver is configured to receive from the sensor device multiple types of measured health data of the wearer at multiple time instances;
the storage unit is configured to store multiple types of health information of the wearer related to the multiple types of measured health data; and
the controller is configured to have the multiple types of health information of the wearer displayed on the display device at multiple time instances, or to initiate the communication to wirelessly transmit the multiple types of health information of the wearer to another device.

13. The device of claim 1, wherein the controller is configured to receive input from the wearer to be displayed on the display device.

14. A device, comprising:
a receiver to receive, from a wearable device on a wearer, health information of the wearer related to measured health data including a skin temperature of the wearer measured by a temperature sensor in contact with the wearer's skin, wherein the skin temperature is measured within a predetermined time window; and
a controller coupled to the receiver and configured to determine a status of the wearer based on the received health information of the wearer.

15. The device of claim 14, wherein the device and the wearable device are in a wireless personal area network (WPAN).

16. The device of claim 14, wherein the health information of the wearer includes a body temperature of the wearer determined based on the skin temperature of the wearer measured by the temperature sensor.

17. The device of claim 14, further comprising:
a storage device coupled to the controller and the receiver, wherein the storage device is configured to store the received health information.

18. The device of claim 14, wherein the controller is configured to alert people within a predetermined range of the status of the wearer.

19. The device of claim 14, wherein the controller is configured to indicate a social distancing preference.

20. The device of claim 14, wherein the controller is further configured to:
send the received health information of the wearer to a server; and
receive commands from the server.
